(19) United States
(12) Patent Application Publication
(10) Pub. No.: US 2004/0259262 A1
(43) Pub. Date: Dec. 23, 2004

(54) Method for determination of aluminum

(76) Inventor: Daido Ishii, Nagoya-shi Aichi (JP)

Correspondence Address: OLIFF & BERRIDGE, PLC, P.O. BOX 19928, ALEXANDRIA, VA, 22320, US

(21) Appl. No.: 10/498,060

(22) PCT Filed: Jan. 22, 2002
(86) PCT No.: PCT/JP02/00416

(30) Foreign Application Priority Data
Dec. 19, 2001 (JP) 2001-385602

(51) Int. Cl. G01N 21/64

(52) U.S. Cl. 436/73; 436/172

(57) ABSTRACT

The measurement of aluminum concentration is accomplished in high sensitivity with HPLC apparatus spread among the quality control divisions of pharmaceutical companies.

The chelating reaction of the sample and lumogallion as the chelating reagent proceeds in the buffer at pH 6 or more. The reaction swiftly proceeds and is completed in about two minutes in room temperature while the conventional method with lumogallion as the chelating reagent as well completes it in about 20 minutes in high temperature of 80° C. The chelate of lumogallion and aluminum is detected by a fluorescent detector in high sensitivity.

### FIELD OF THE INVENTION

The present invention relates to a method for measuring a content of aluminum in a sample by chromatography, and to a chelating reagent for the method.

### DESCRIPTION OF RELATED ART

Flameless atomic absorption spectrometry, ICP-AES (Inductively coupled plasma-auger electron spectroscopy) and ICP-MS (Inductively coupled plasma-mass spectroscopy) are usually employed for measurement of aluminum in pharmaceuticals. During ten years and more, they have tried to measure aluminum in an injection in the United States, and employed the flameless atomic absorption spectrometry in many of the cases. It is requisite to measure the content of aluminum in pharmaceuticals, especially, injections, because a regulation of the content of aluminum is to be enforced in the U.S. and Japan.

Use of the flameless atomic absorption spectrometry, ICP-AES and ICP-MS substantially allows the measurement of the aluminum content in a high sensitivity, such as ppb or ppt level, if the sample is pure. However, it is difficult to measure very small amount of aluminum in a complex matrix, such as the injection, by them. The measurement requires a pretreatment and results in low sensitivity and reproducibility. (S. Nomoto et al., Biomed. Res. Trace Elements, Vol. 8, 245 (1997).)

Furthermore, the flameless atomic absorption spectrometry, ICP-AES and ICP-MS require ventilating equipment, piping of gas and so on. For this reason, it is very difficult to set up and work them in small quality control facilities. And they do not allow an unattended night operation required in quality control because they requires control of dangerous matters, such as inflammable or toxic gases, an atomization furnace, plasma. (“Measurement of Aluminum in Injection by Fluorescent HPLC” The Japan Society for Analytical Chemistry, 50^{th} Year Meeting Abstracts, p. 133.”

Other method of HPLC (High performance liquid chromatography) than the flameless atomic absorption spectrometry, ICP-AES and ICP-MS is known, and announces methods for measuring the content of aluminum in a biological component, such as blood serum and urine, and in seawater. HPLP separates and detects aluminum chelates generated in a reaction of aluminum and chelating reagent. Known chelating reagents include DHAB (2,2′-dihydroxyazobenzene) (E. Kaneko et al., Analytical Chemistry 63 (1991) 2219-2222), lumogallion (Y. Suzuki et al., Analyst 114 (1989) 839-842; J. Wu et al., Journal of Chromatography B 663 (1995) 247-253) and 8-quinolinol (M. Sato et al., Journal of Chromatography A 789 (1997) 361-367).

In a conventional method using lumogallion as the chelating reagent, aluminum chelates are synthesized by reaction in a weak acid liquid of pH 4-5 in which aluminum chelates are detected in high sensitivity by a fluorescent detector (Nishikawa et al., Analytical Chemistry, Vol. 16, 692-697 (1967)). It requires additional handlings as requiring sufficient time of about 20 mitutes for reaction or heating the measurement mixture to about 70° C. in order to facilitate a reaction rate of chelating. It is a disadvantage in measurement for quality control. Including this, there are known disadvantages in the conventional method using DHAB as the chelating reagent: required heating to about 70° C. and sufficient time of about 20 mitutes for chelating reaction; low sensitivity and selectivity in a light absorption method; prevented measurement of aluminum if a large quantity of iron is in the sample owing to a peak of iron close to a peak of aluminum; and unavoidability of an influence of an interfering substance, such as deferoxamine (DFO). Only one kit for detecting aluminum by chromatography for sale is that (Dojindo Laboratories, Kumamoto) using 8-quinolinol as the chelating reagent. No kit using lumogallion or DHAB as the chelating reagent is for sale. The kit using 8-quinolinol includes the chelating reagent, a chelate forming solution (a buffer) and an eluant.

Aluminum in some injections is hard to be measured with the kit using 8-quinolinol. Substantial equality in fluorescent wavelengths of the aluminum chelate and the vitamin B₂ prevents from distinguishing the signals of the fluorescent wavelengths and measuring the content of aluminum in a vitamin B₂ preparation. And indistinguishability of a signal of the aluminum chelate from a signal of a matrix prevents from measurement of the content of aluminum in a lipid emulsion.

Dilution of the measurement sample or the reaction mixture is required in order to reduce the signal of the matrix and an influence or contamination of aluminum from the external environment. But low fluorescent sensitivity prevents measurement of the content of aluminum using 8-quinolinol as the chelating reagent if diluted.

The eluant, or the mobile phase, containing an organic solvent of high concentration of 15 percent or more causes waste disposal at considerable cost because the measurement using 8-quinolinol produces dangerous waste.

It is therefore an object of the present invention to provide a method for measuring the content of aluminum in the sample swiftly and in high sensitivity.

### SUMMARY OF THE INVENTION

The object indicated above may be achieved according to a first aspect of the invention, which provides a method for measuring a content of aluminum by detecting aluminum chelate with a fluorescent detector, wherein the aluminum chelate is generated by reaction in a reaction mixture of a sample containing aluminum and a chelating reagent containing lumogallion, and the reaction mixture has a pH value of six or higher.

The object indicated above may be achieved according to a second aspect of the invention, which provides a method for measuring a content of aluminum by detecting aluminum chelate with a fluorescent detector, wherein the aluminum chelate is generated by reaction in a reaction mixture of a sample containing aluminum and a chelating reagent containing lumogallion and separated from the reaction mixture by liquid chromatography, and the reaction mixture has a pH value of six or higher.

The object indicated above may be achieved according to a third aspect of the invention, which provides a method for measuring a content of aluminum according to the second aspect of the invention, characterized by that a concentration of an organic solvent in a mobile phase in the liquid chromatography is five percent or less.

The object indicated above may be achieved according to a fourth aspect of the invention, which provides a method for measuring a content of aluminum according to any of the first, second and third aspects of the invention, characterized by that the reaction mixture contains one of reagents for preparing a buffer, which one of the reagents are selected from a group of 2-morpholinoethanesulfonic acid (MES), piperazine-1,4-bis (2-ethanesulfonic acid) (PIPES), (2-[4-(2-hydroxyethyl)-1-piperazinyl] ethanesulfonic acid (HEPES), N-tris (hydroxymethyl) methyl-2-aminoethanesulfonic acid (TES), (3-[4-(2-hydroxyethyl)-1-piperazinyl] propanesulfonic acid (EPPS), N-cyclohexyl-2-aminoethanesulfonic acid (CHES) and 3-morpholinopropanesulfonic acid (MOPS).

The object indicated above may be achieved according to a fifth aspect of the invention, which provides a method for measuring a content of aluminum according to any of the first, second, third and fourth aspects of the invention, characterized by that the reaction mixture is the sample diluted 10-fold or more.

The object indicated above may be achieved according to a sixth aspect of the invention, which provides a method for measuring a content of aluminum according to any of the first, second, third, fourth and fifth aspects of the invention, characterized by that the sample is a pharmaceutical.

The object indicated above may be achieved according to a seventh aspect of the invention, which provides a method for measuring a content of aluminum according to the sixth aspect of the invention, characterized by that the pharmaceutical contains vitamin B₂.

The object indicated above may be achieved according to a eighth aspect of the invention, which provides a method for measuring a content of aluminum according to the sixth aspect of the invention, characterized by that the pharmaceutical contains lipid emulsion.

According to the feature of the first aspect of the invention, measurement of the content of aluminum in the sample in high sensitivity is achieved by detecting the chelate of lumogallion and aluminum with the fluorescent detector using the chelating reagent containing lumogallion. And chelate forming reaction is completed in about two minutes because it proceeds in the mixture of the pH value of six or higher and consequently the reaction rate substantially increases at a room temperature of 25° C. The method is suitable for measurement in a routine for quality control and so forth.

The measurement in the first aspect of the invention by a batch or flow injection method may be conducted for the content of aluminum by detecting the aluminum chelate with the fluorescent detector without the separation of the reaction mixture. However, some samples contain a component detected by the fluorescent detector other than the chelate of lumogallion and aluminum. The method according to the second aspect of the invention may provide substantially accurate measurement of the content of aluminum even in such samples by detecting the chelate with the fluorescent detector after the separation of the chelate of lumogallion and aluminum from the reaction mixture by liquid chromatography.

The liquid chromatography according to the method in the second aspect of the invention may be replaced by a reversed-phase liquid chromatography, which a concentration of an organic solvent in a mobile phase is five percent or less. This causes reduced waste of the organic solvent, waste disposal at low cost and reduced influences on the environment.

While the buffer is generally added to the reaction mixture for chelating reaction at a constant pH, it is usually contaminated with aluminum in a production process. The method according to the fourth aspect of the invention may provide with measurement of the content of aluminum in higher sensitivity with reduction of a blank value by any of MES, PIPES, HEPES, TES, EPPS, CHES and MOPS as a reagent for preparing the buffer.

The method according to the fifth aspect of the invention allows detection of the aluminum chelate even in the sample diluted 10-fold or more owing to its high sensitivity in detection according to the method of the first aspect of the invention. The Dilution of 10-fold or more reduces the influence of the matrix and allows measurement of the content of aluminum in higher sensitivity.

The method according to the sixth aspect of the invention may measure the content of aluminum in a pharmaceutical. The difference of fluorescent wavelengths between the aluminum chelate and vitamin B₂ provide complete distinction between each of the signals. The fluorescent wavelength of the aluminum chelate generated by the reaction of lumogallion and aluminum is 574 nm. The method according to the seventh aspect of the invention may measure the content of aluminum in a pharmaceutical containing vitamin B₂, in contrast to the difficulty in the method using 8-quinolinol as the chelating reagent, which the excitation and fluorescent wavelengths of 8-quinolinol are substantially the same as those of vitamin B₂ respectively. The method according to the eighth aspect of the invention may measure the content of aluminum in a pharmaceutical containing lipid emulsion for the difference between the fluorescent wavelengths of the aluminum chelate and the matrix component of lipid emulsion.

### BRIEF DESCRIPTION OF THE DRAWINGS

FIG. 1 illustrates a block diagram of a general high performance liquid chromatograph apparatus according to the invention;

FIG. 2 illustrates a calibration graph of the first embodiment;

FIG. 3 illustrates a graph of reproducibility of measurement;

FIG. 4 illustrates chromatograms by the method for measuring the content of aluminum: (a) is a chromatogram of a hyperalimentation retentive solution; and (b) is a chromatogram of a multi amino acid injection including xylitol;

FIG. 5 illustrates a graph of fluorescence intensity of the aluminum chelate which is chelated in buffers of different pH;

FIG. 6 illustrates chromatograms of a vitamin B₂ preparation: (a) is a chromatogram by the method according to the invention; and (b) is a chromatogram by the method using 8-quinolinol as the chelating reagent;

FIG. 7 illustrates chromatograms of lipid emulsion: (a) is a chromatogram by the method according to the invention; and (b) is a chromatogram by the method using 8-quinolinol as the chelating reagent;

FIG. 8 illustrates chromatograms of the vitamin preparation: (a) is a chromatogram without dilution by the method according to the invention; (b) is a chromatogram with the sample diluted 10-fold or more by the method according to the invention; and (c) is a chromatogram with the sample diluted 10-fold or more by the method using 8-quinolinol as the chelating reagent; and

FIG. 9 illustrates a chromatogram of 30% glucose solution using the mobile phase solvent without an organic solvent by the method according to the invention.

### DETAILED DESCRIPTION OF EXEMPLARY EMBODIMENTS

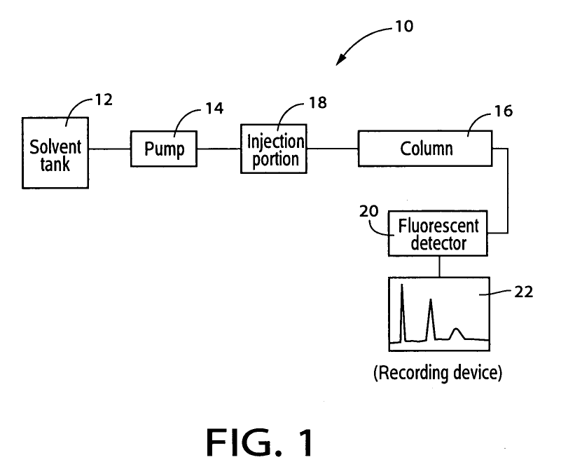

Hereinafter, there will be described a method for measuring the content of aluminum embodying the invention, by reference to the drawings. FIG. 1 illustrates a block diagram of a general high-performance liquid chromatograph apparatus (hereinafter referred to as HPLC) according to the invention.

In FIG. 1, a mobile phase solvent (eluant) previously stored in a solvent tank 12 is introduced to a column 16 by a pump 14 at a predetermined speed such as about 1.0 mL/min.

A solvent suitable for retention and elution of the lumogallion-aluminum chelate is employed for the mobile phase. It may be the buffer or a mixture of the buffer and the organic solvent. It may be the ‘third reagent’ (referred to later), or 2-propanol of 25 w/v %, 0.1 mol/L acetic acid buffer (pH 4.6), and the concentration of 2-propanol may be over or below 25 w/v %. Other organic solvent than 2-propanol or no organic solvent may be employed. The pH of the buffer and the reagent for preparation of the buffer may vary as far as they are suitable for retention and elution of the lumogallion-aluminum chelate.

In an injection portion 18, the prepared reaction mixture of the sample and the chelating reagent is injected to the column 16 through an injector (not shown). The reaction mixture may be automatically prepared by an apparatus. The column 16 may be formed of a stainless steel tube of 4.6 mm in internal diameter and 150 mm in length. The reaction mixture injected from the injection portion 18 is introduced to the fluorescent detector 20 after the separation in the column 16. The result by the fluorescent detector 20 is recorded in a recording device 22.

The sample may be material of a pharmaceutical, a pharmaceutical, a plant tissue, an animal tissue, material of health food, health food, material of drinking water, drinking water, material of cosmetics, cosmetics, tea, alcoholic drink, tap water, environmental water, seawater, lake water, river water, industrial waste water, industrial water, a research reagent, raw material of industry, antibody, vaccine formed from antigen, serum, urine, plasma, blood, body fluid of a human being or an animal such as semen, sweat, tear, ascites fluid or amnionic fluid. The pharmaceutical includes: one for circulatory system such as a cardiac stimulant, an antianginal drug, an antiarrhythmic agent, a diuretic, an antihypertensive agent, a vasodilator and a hypertensor; one for alimentary system such as a stomachic, a digestant, a peptic ulcer agent, a cathartic, an antidiarrhoic, an antiflatuent and a cholagogue; one for respiratory system such as an anapnoic, a bronchodilator, an expectorans and an antiphlegmy agent; one for blood system such as a hematinic, a hemostat and an antithrombotic drug; one for nerve system such as a hypnotic, an abirritant, a analgesic, an anticonvulsant, an anti cerebral circulatory disorder drug, an antiparkinsonism drug, a muscle relaxant, an antiemetic and an antivertigo agent; one for psychiatry such as an antipsychotic, an antianxiety agent, an antidepressant and an autonomic drug; one for endocrine and metabolic system such as an insulin preparation, an oral antidiabetic, an antidiabetic drug such as a postcibal hyperglycemia amelioration drug, a hypolipidemic drug, an antarthritic, hyperuricacidemia drug, a bone and calcium metabolism drug, a thyroid hormone preparation, an estrogen preparation, an androgen preparation and other hormone preparations; one for inflammatio, allergy and immunosuppressant such as an adrenocorticosteroid drug, a nonsteroidal anti-inflammatory drug, an antiphlogistic enzyme drug, an antirheumatic, an antihistamic agent, an antiallergic agent and an immunosuppressant; an anticancer agent such as a alkylating agent, an antimetabolite, a carcinostatic antibiotic, a plant alkaloid and a biological response modifier (BRM); one against pathogenic microorganisms such as an antibacterial agent, an antifungal agent, an antiviral agent, an antiparasitic agent and an antiprotozoan agent; a chinese herbal remedy, an injection, an infusion, a dialysate, an antibiotic and a blood product. The infusion includes a carbohydrate transfusion, an amino acid preparation, a dextran transfusion, a lipid emulsion, a protein amino acid compounding agent, an electrolyte transfusion, a hyperalimentation, an electrolyte replenisher, a heparin preparation. The dialysate includes a peritoneal dialysis fluid and an artificial kidney dialysate. The hematinic includes a chalybeate for injection, a vitamin B₂ preparation, a vitamin B₁₂ preparation, folic acid, a vitamin B₆ preparation, an erythropoietin preparation and a colony stimulating factor (CSF) preparation. The antibiotic includes the preparations of penicillins, cephem, monobactam, carbapenem, aminoglycoside, macrolide, tetracycline, pyridone carboxylic acid and peptide. The antiviral agent includes an antiherpesviral agent, an anticytomegaloviral agent, an anti-HIV agent and an anti-influenza viral agent. The blood product includes fractionated products.

Preferably it is the sample in a form of liquid. The sample is suitably made by diffluence or extraction if it is not in a form of liquid.

First Embodiment: Preparation of Calibration Graph

[Preparation of Reagent and Sample]

The reagent is prepared as follows.

Preparation of Chelate Reagent (First Reagent)

The solution of 1 m mol/L lumogallion and 1 mol/L hydrochloric acid was prepared from lumogallion (a chelating reagent for analysis) [Dojindo Laboratories], hydrochloric acid (of ultra-high purity) [Kanto Kagaku] and ultrapure water (of ultra-high purity) [Kanto Kagaku]. The reagent of ultra-high purity is manufactured so as to include aluminum and other trace elements of less than ppt (parts per trillion) level. The addition of hydrochloric acid causes the mixture of the chelating reagent and the sample to be acid and causes aluminum in the sample to be ionized. The hydrochloric acid may be replaced by nitric acid or other acids. A neutral chelating reagent may replace hydrochloric acid if any acid is previously added in the sample. The organic solvent may be added in the chelating reagent.

Preparation of Buffer (Second Reagent)

A 0.5 mol/L MES buffer (pH 7.0) was prepared from MES (2-morpholinoethanesulfonic acid, monohydrate) (a reagent for analysis) [Dojindo Laboratories], sodium hydrate (of ultra-high purity) [Kanto Kagaku] and ultrapure water (of ultra-high purity) [Kanto Kagaku].

Preparation of Eluant (Third Reagent)

A 2-propanol of 25 w/v %, 0.1 mol/L acetic acid eluant (pH 4.6) was prepared from 2-propanol (HPLC grade) [Kanto Kagaku], sodium acetate (a reagent chemical) [Wako Pure Chemical Industries, Ltd.], hydrochloric acid (of ultra-high purity) [Kanto Kagaku] and ultrapure water (of ultra-high purity) [Kanto Kagaku].

Preparation of Samples for Calibration Graph

An aluminum standard solution for atomic absorption (for analysis) was diluted 1:9 in volume with a 0.1 mol/L nitric acid solution prepared from ultra-high pure nitric acid (of ultra-high purity) [Kanto Kagaku] and ultrapure water (of ultra-high purity) [Kanto Kagaku]. The samples having the respective aluminum concentration of 0, 5, 10, 15, 20, 25, 30, 35, 40, 45 and 50 μg/L were prepared.

[Measurement of Aluminum Concentration]

The chelating reagent, or the first reagent, of 50 μL was in a 1.5 mL sample vial, the sample for the calibration graph of 300 μL was added therein, and the mixture was swiftly stirred and mixed. The buffer, or the second reagent, of 600 μL was added therein and the mixture was swiftly stirred and mixed again. The addition of the second reagent caused the reaction mixture to be neutral and consequently aluminum ions swiftly react with lumogallions to swiftly generate aluminum chelates. The reaction mixture stood at room temperature (25° C.) for five minutes and the mixture of 20 μL was injected into the injection portion 18 of the HPLC 10 and measured. The measurement of the respective samples for the calibration graph was conducted twice. Especially the measurement of the respective samples of the aluminum concentration of 0, 5 and 10 μg/L was conducted five times for reproducibility.

[Condition for HPLC Measurement]

Flow Rate: 1.0 mL/min

Column: Develosil LAL 4.6 mm i.d.×150 mm (Nomura Chemical Co., Ltd.)

Temperature of Column: 40° C.

Wavelength: Ex. (Excitation wavelength)=505 nm, Em. (Detection wavelength)=574 nm

[Result of Measurement]

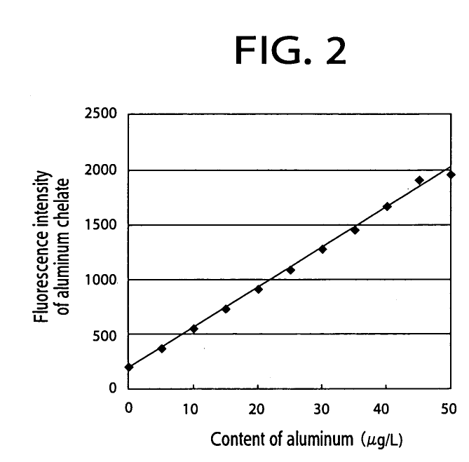

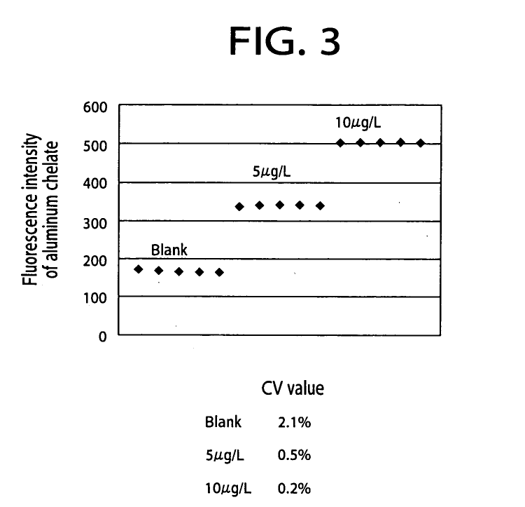

FIG. 2 shows the quantitative determination at least between 0 and 50 μg/L. FIG. 3 shows the considerable reproducibility. The CV (Coefficient of variation) value in FIG. 3 equals a standard deviation (divided by an average.

Second Embodiment: Measurement of Aluminum Content in Infusion

[Preparation of Reagent and Sample]

The same reagents, the first, second and third reagents, for the chelating reagent, the buffer and the eluant as ones for the first embodiment were used. Each of UNICALIQ^{(R)}N, a maintenance medium for hyperalimentation, (dextrose 17.5%, amino acid 3.0%, L-sodium lactate 0.4%) [Terumo Corporation] and TERUAMINO^{(R)}12X, a multi amino acid injection including xylitol, (xylitol 5%) [Terumo Corporation] was used by itself as a sample. UNICALIQ^{(R)} and TERUAMINO^{(R)} are the registered trademarks of Terumo Corporation.

A Standard Substance of River Water (JAC0031, JAC0032) of the Japan Society for Analytical Chemistry was used by itself as a calibrator, an internal standard sample. These are the standard substances having the certified values of the aluminum contents by ICP-AES, ICP-MS, GF-AAS (graphite furnace-atomic absorption spectrometry) and fluorescent method. The JAC0031 is certified as including aluminum of 13.4±0.7 μg/L and the JAC0032 is certified as including aluminum of 61±2 μg/L.

[Measurement of Aluminum Concentration]

(1) Measurement of Aluminum Concentration According to the Invention

The chelating reagent, the first reagent, of 50 μL was in a 1.5 mL sample vial, the measurement sample of 300 μL was added therein, and the mixture was swiftly stirred and mixed. The buffer, the second reagent, of 600 μL was added therein and the mixture was swiftly stirred and mixed again. The reaction mixture stood at room temperature (25° C.) for five minutes, and then the mixture of 20 μL was injected into the injection portion 18 of the HPLC 10 and measured. The measurement samples were respectively measured three times in succession.

(2) Measurement of Aluminum Concentration Using 8-quinolinol as Chelate Reagent

The aluminum measuring kit (Dojindo Laboratories, Kumamoto) using 8-quinolinol as the chelating reagent was used for a reference value in a recommended practice. The chelating reagent solution of 50 μL including 8-quinolinol and hydrochloric acid was in a 1.5 mL sample vial, the measurement sample of 150 μL was added therein and the mixture was swiftly stirred and mixed. The buffer of pH 7.5 of 400 μL was added therein and the mixture was swiftly stirred and mixed again. The reaction mixture stood at room temperature (25° C.) for ten minutes, and then the reaction mixture of 200 μL was injected into the injection portion 18 of the HPLC 10 and measured. The eluant was the solution of pH 7.0 including surfactant.

[Condition for HPLC Measurement]

(1) Measurement of Aluminum Concentration According to the Invention

Flow Rate: 1.0 mL/min

Column: Develosil LAL 4.6 mm i.d.×150 mm (Nomura Chemical Co., Ltd.)

Temperature of Column: 40° C.

Wavelength: Ex. =505 nm, Em. =574 nm

(2) Measurement of Aluminum Concentration Using 8-quinolinol as Chelate Reagent

Flow Rate: 1.0 μL/min

Column: Dedicated column for aluminum analysis 4.6 mm i.d.×50 mm (Shino-Test Corporation)

Temperature of Column: 25° C.

Wavelength: Ex. =370 μm, Em. =504 nm

[Result of Measurement]

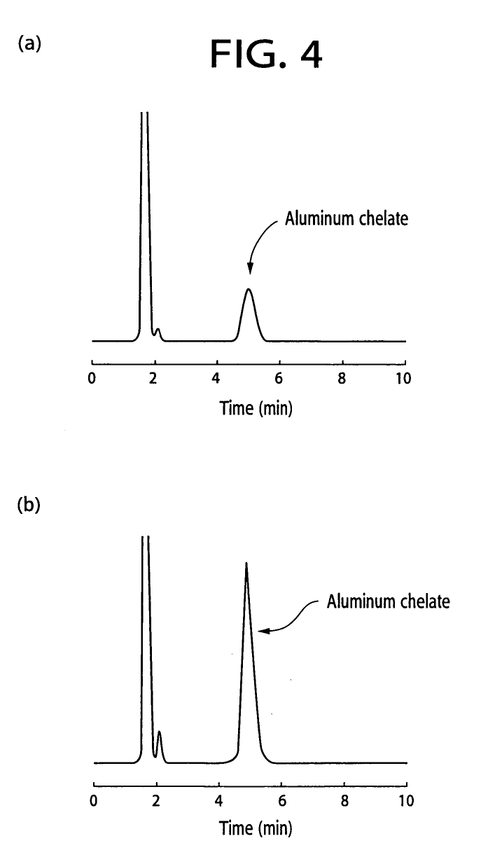

FIG. 4 shows no other peaks near the peak of the aluminum chelate in the hyperalimentation maintenance medium and the multi amino acid injection including xylitol measured by the method according to the invention. As shown in Table 1, the measured values are substantially equal to the reference values measured using 8-quinolinol as the chelating reagent within a range of measurement error.

TABLE 1
Concentrations Measured by Method According to the Invention
and Method Using 8-quinolinol

| | Measured Value | Reference |
|---|---|---|
| | (The Invention) | Value |
| Hyperalimentation | 4.5 ppb | 4.3 ppb |
| Maintenance Medium | | |
| Multi Amino Acid Injection | 21 ppb | 19 ppb |
| Including Xylitol | | |

Third Embodiment: Comparing Method Using 8-quinolinol as Chelate Reagent to Method According to the Invention in Sensitivity

[Preparation of Reagent and Sample]

The same reagents, the first, second and third reagents, for the chelating reagent, the buffer and the eluant as ones for the first embodiment were used. The Standard Substance of River Water (JAC0031) of the Japan Society for Analytical Chemistry was used by itself as a measurement sample.

[Measurement of Aluminum Concentration]

The aluminum concentration according to the invention was measured by the same method of the second embodiment. The aluminum concentration using 8-quinolinol as the chelating reagent was also measured by the same method of the second embodiment.

[Result of Measurement]

As shown in Table 2, the measurement according to the invention was about five times the measurement using 8-quinolinol as the chelating reagent in sensitivity. As shown in Table 3, the amount of the measurement sample in the injection of the measurement using 8-quinolinol as the chelating reagent is about eight times that according to the invention. Consequently, the measurement according to the invention is estimated to be about 40 times the measurement using 8-quinolinol as the chelating reagent in sensitivity. This allows the measurement of the injection diluted about 30-fold or more comparing to the measurement using 8-quinolinol, and allows less influences of aluminum derived from the external environment.

TABLE 2
Peak Area of Chromatogram

| | Measurement | Measurement |
|---|---|---|
| | According to | Using |
| | the Invention | 8-quinolinol |
| Blank Value | 177.8 | 13.40 |
| JAC0031 Measured Value | 657.7 | 107.3 |
| Difference | 479.9 | 93.9 |
| | (5.11) | (1.00) |

TABLE 3
Amount of Injected Sample

| | | Measurement Using |
|---|---|---|
| | The Invention | 8-quinolinol |
| Amount of JAC0031 | 300 μL | 150 μL |
| Amount of 1^{st} Reagent | 50 μL | 50 μL |
| Amount of 2^{nd} Reagent | 600 μL | 400 μL |
| Total | 950 μL | 600 μL |
| Injected Amount | 20 μL | 200 μL |
| Amount of JAC0031 | 6.3 μL | 50 μL |
| in Injection | (1.00) | (7.94) |

Fourth Embodiment: Relationship between Reaction Rate and Reaction pH

[Preparation of Reagent and Sample]

The same reagents, the first and third agents, for the chelating reagent and the eluant as ones for the first embodiment were used. The Standard Substance of River Water (JAC0031) of the Japan Society for Analytical Chemistry was used by itself as a measurement sample.

Buffers of pH 4.0, 5.0, 6.0, 6.5, 7.0, 8.0 and 9.0 for chelating reaction were prepared as follows.

Buffers of pH 4.0 and 5.0

Buffers of 0.5 mol/L acetic acid of pH 4.0 and 0.5 mol/L acetic acid of pH 5.0 were prepared from sodium acetate (a reagent chemical) [Wako Pure Chemical Industries, Ltd.], sodium hydrate (of ultra-high purity) [Kanto Kagaku] and ultrapure water (of ultra-high purity) [Kanto Kagaku].

Buffers of pH 6.0, 6.5 and 7.0

Buffers of 0.5 mol/L MES of pH 6.0, 0.5 mol/L MES of pH 6.5 and 0.5 mol/L MES of pH 7.0 were prepared from MES (a reagent for analysis) [Dojindo Laboratories], sodium hydrate (of ultra-high purity) [Kanto Kagaku] and ultrapure water (of ultra-high purity) [Kanto Kagaku].

Buffer of pH 8.0

A buffer of 0.5 mol/L BES of pH 8.0 was prepared from BES (N,N-bis (2-hydroxyethyl)-2-aminoethanesulfonic acid) (a reagent for analysis) [Dojindo Laboratories], sodium hydrate (of ultra-high purity) [Kanto Kagaku] and ultrapure water (of ultra-high purity) [Kanto Kagaku].

Buffer of pH 9.0

A buffer of 0.5 mol/L CHES of pH 9.0 was prepared from CHES (N-cyclohexyl-2-aminoethanesulfonic acid) (a reagent for analysis) [Dojindo Laboratories], sodium hydrate (of ultra-high purity) [Kanto Kagaku] and ultrapure water (of ultra-high purity) [Kanto Kagaku].

[Measurement of Aluminum Concentration]

The chelating reagent, the first reagent, of 50 μL was in a 1.5 mL sample vial, the measurement sample of 300 μL was added therein and the mixture was swiftly stirred and mixed. One of the above buffers of pH 4.0-9.0 of 600 μL was added therein and the mixture was swiftly stirred and mixed again. The reaction mixture stood at room temperature (25° C.) for five minutes and the mixture of 20 μL was injected into the injection portion 18 of the HPLC 10 and measured. The condition for HPLC measurement is the same as that in the second embodiment. For measuring blank values, the chelating reagent, the first reagent, of 50 μL was in a 1.5 mL sample vial and one of the above buffers of pH 4.0-9.0 of 600 μL was added therein and the mixture was swiftly stirred and mixed. The reaction mixture stood at room temperature (25° C.) for five minutes and the mixture of 20 μL was injected into the injection portion 18 of the HPLC 10 and measured.

[Result of Measurement]

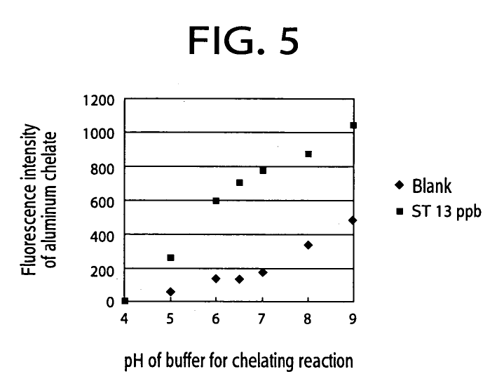

As shown in FIG. 5, no signal of the aluminum chelate was detected in the measurement using the buffer of pH 4.0. While the signals of the aluminum chelate was detected in the measurement using the buffer of pH 5.0, low signal intensity of the aluminum chelate was presumed to lead to the incomplete chelating reaction. Presuming that the difference in the signal intensity between the blank value and the value of the measurement sample was reflective of the aluminum concentration, the signal intensities derived from aluminum in the measurement samples were considered to substantially equal one another in the measurement using the buffers of pH 6.0 and more.

Fifth Embodiment: Comparing Method Using 8-quinolinol as Chelate Reagent to Method According to the Invention in Measurement of Preparation Including Vitamin B₂

[Preparation of Reagent]

The same reagents, the first, second and third reagents, for the chelating reagent, the buffer and the eluant as ones for the first embodiment were used.

[Preparation of Measurement Sample]

A K.C.L. injection (No. 1) including vitamin B₂ of 300 mg/L (potassium chloride 15 w/v % [Maruishi Pharmaceutical Co., Ltd.]) was used by itself.

The Standard Substance of River Water (JAC003 1, JAC0032) of the Japan Society for Analytical Chemistry was used by itself as a calibrator.

[Measurement of Aluminum Concentration]

The aluminum concentration according to the invention was measured by the same method as in the second embodiment. The aluminum measuring kit using 8-quinolinol as the chelating reagent was used for a reference value in a recommended practice. Both of these were measured in the same condition of HPLC measurement as in the second embodiment.

[Result of Measurement]

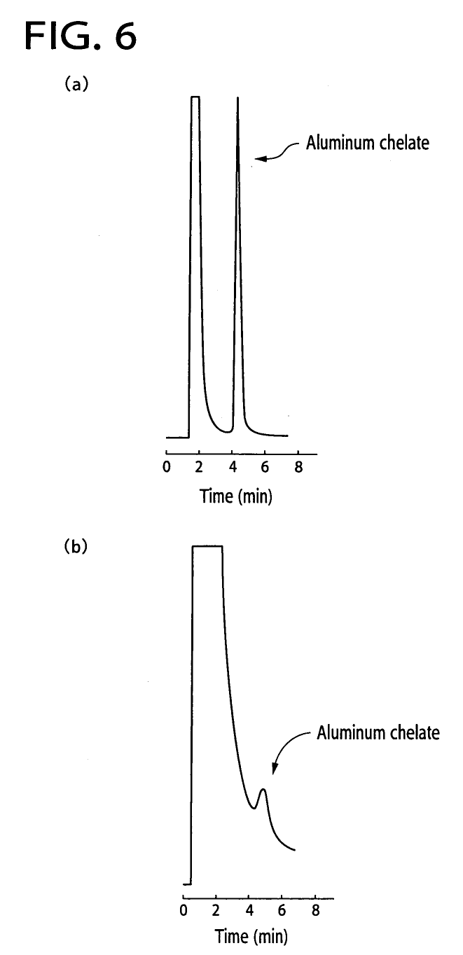

As shown in FIG. 6(a), the completely separated peaks derived from the aluminum chelate and the matrix mean that it is possible to measure the aluminum concentration. In contrast, it is difficult to measure the aluminum concentration in the measurement by the method using 8-quinolinol as the chelating reagent because the peak of the aluminum chelate is not conspicuously projected, that is, not completely independently plotted and it is almost embedded in the peak of the matrix as shown in FIG. 6(b).

Sixth Embodiment: Comparing Method Using 8-quinolinol as Chelate Reagent to Method According to the Invention in Measurement of Lipid Emulsion

[Preparation of Reagent]

The same reagents, the first, second and third reagents, for the chelating reagent, the buffer and the eluant as ones for the first embodiment were used.

[Preparation of Measurement Sample]

An INTRAFAT^{(R)}, a lipid emulsion, (soybean oil 10%) [Nihon Pharmaceutical Co., Ltd.]) was used by itself. INTRAFAT^{(R)} is the registered trademark of Nihon Pharmaceutical Co., Ltd.

The Standard Substance of River Water (JAC003 1, JAC0032) of the Japan Society for Analytical Chemistry was used by itself as a calibrator.

[Measurement of Aluminum Concentration]

The aluminum concentration according to the invention was measured by the same method as in the second embodiment. The aluminum measuring kit using 8-quinolinol as the chelating reagent was used for a reference value in a recommended practice. Both of these were measured in the same condition of HPLC measurement as in the second embodiment.

[Result of Measurement]

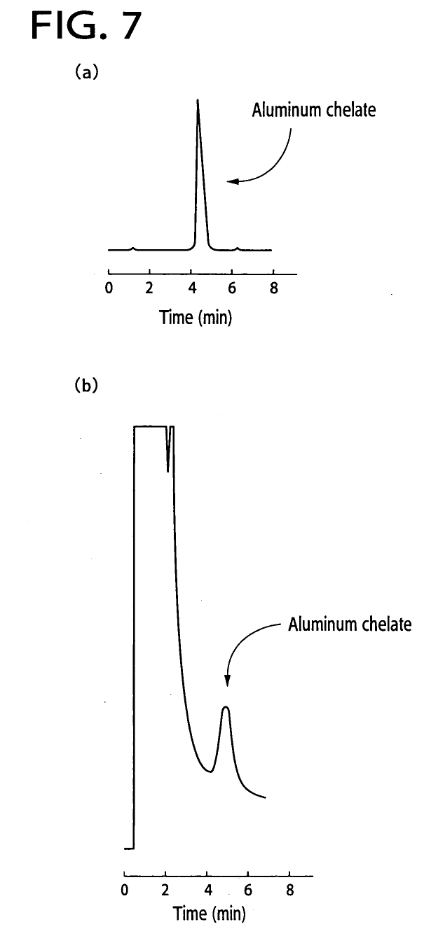

As shown in FIG. 7(a), hardly detected peaks derived from the matrix cause the measurement of the aluminum concentration in high sensitivity. Batch measurement or a flow injection method may be adopted for measurement of the aluminum concentration in such a case as hardly detected components other than the aluminum chelate require no separation.

In contrast, it is difficult to measure the aluminum concentration by the method using 8-quinolinol as the chelating reagent because the peak of the aluminum chelate is not conspicuously projected, that is, not completely independently plotted and it is almost embedded in the peak of the matrix in FIG. 7(b).

Seventh Embodiment: Measurement of Aluminum Concentration in Vitamin Preparations by Dilution Method

[Preparation of Reagent]

The same reagents, the first, second and third reagents, for the chelating reagent, the buffer and the eluant as ones for the first embodiment were used.

[Preparation of Measurement Sample]

Neo M.V.I.-9 (including riboflavin sodium phosphate (vitamin B²) as riboflavin of 3.6 mg in 5 mL) [SSP Co., Ltd.]) was used by itself.

The Standard Substance of River Water (JAC0031, JAC0032) of the Japan Society for Analytical Chemistry was used by itself as a calibrator.

[Measurement of Aluminum Concentration]

(1) Measurement of Aluminum Concentration According to the Invention

The chelating reagent, the first reagent, of 50 μL was in a 1.5 mL sample vial, the measurement sample of 300 μL was added therein, and the mixture was swiftly stirred and mixed. The buffer, the second reagent, of 600 μL was added therein and the mixture was swiftly stirred and mixed again. The measurement sample had been diluted about three-fold at that time. The reaction mixture stood at room temperature (25° C.) for two minutes and the sample was diluted about 30-fold to be 9.5 mL with ultrapure water. The diluted mixture of 20 μL was injected into the injection portion 18 of the HPLC 10 and measured. The measurement of it was conducted ten times in succession. For comparison, the reaction mixture prior to dilution with ultrapure water, that is, the reaction mixture with the measurement sample diluted about three-fold was measured in the same way.

(2) Measurement of Aluminum Concentration Using 8-quinolinol as Chelate Reagent

The aluminum measuring kit (Dojindo Laboratories, Kumamoto) using 8-quinolinol as the chelating reagent was used in a recommended practice. The measurement sample had been diluted about four-fold at that time. Furthermore, the reaction mixture was diluted about 10-fold with ultrapure water. It means that the measurement sample was diluted about 40-fold. The mixture of 200 μL prior to dilution with ultrapure water, that is, the mixture with the measurement sample diluted about four-fold, and the mixture diluted with ultrapure water, that is, the mixture of 200 μL with the measurement sample diluted about 40-fold were respectively injected into the injection portion 18 of the HPLC 10 and measured.

[Condition for HPLC Measurement]

The aluminum concentrations according to the invention and using 8-quinolinol as the chelating reagent were measured in the same condition as in the second embodiment.

[Result of Measurement]

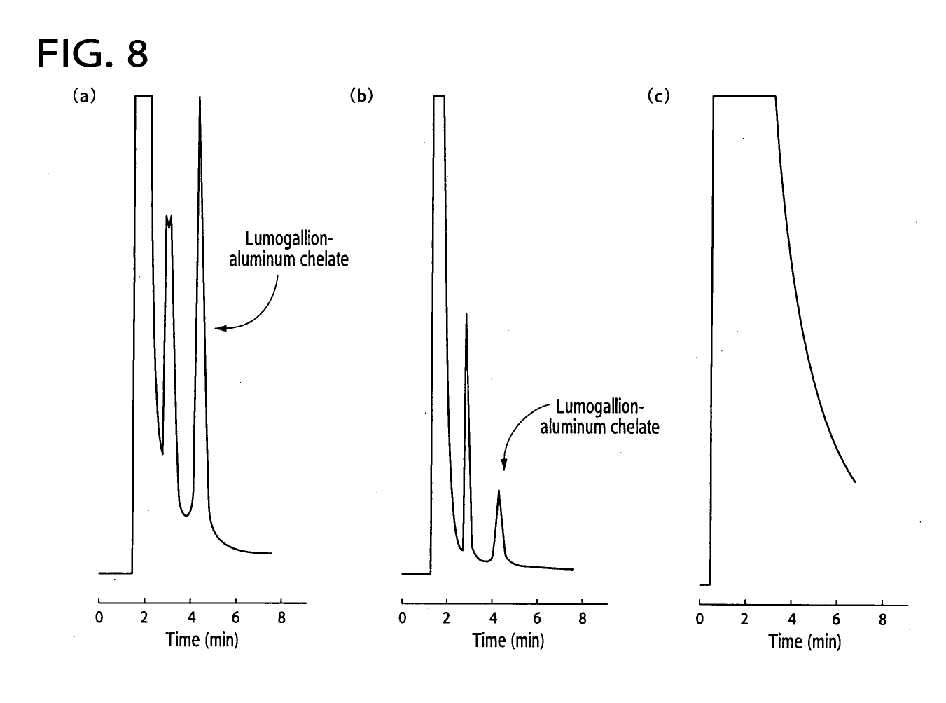

According to the invention, in the case of the mixture with the measurement sample diluted about three-fold, the aluminum concentration calculated from the peak area in FIG. 8(a) does not result in very high reliability because the signal derived from the aluminum chelate is partly ovelapped with that derived from the matrix as shown in FIG. 8(a). The possible detection of the peak of the aluminum chelate in the about 30-fold diluted measurement sample allows the measurement of the aluminum concentration in ppb level as shown in FIG. 8(b) even if the measurement sample is diluted about 30-fold. Consequently, it is also possible to measure the aluminum concentration of the measurement sample diluted in the lower dilution ratio than 30-fold, such as about 10-fold. Furthermore, the about 30-fold dilution allows accurate measurement of the aluminum concentration because the signal of the aluminum chelate is sufficiently separately plotted from other signals. The aluminum concentration provided from the signal intensity in FIG. 8(b) was 2.1 ppb.

FIG. 8(c) is the chromatogram of the mixture with the sample diluted about 40-fold by the method using 8-quinolinol as the chelating reagent. The peak of the aluminum chelate is completely embedded in the peak derived from the matrix and is not distinguished from it. On the basis of the measurement of the fifth embodiment, the aluminum chelate should be in about five minutes passed. In order to define the peak of the aluminum chelate, the reduction of the amount of the matrix components is required with further dilution. However, the method using 8-quinolinol as the chelating reagent is lower than the method according to the invention in sensitivity as described in the third embodiment (for comparing sensitivity) and further dilution does not define the peak of the aluminum chelate. Consequently, the method using 8-quinolinol as the chelating reagent does not allow the measurement of the aluminum concentration in Neo M.V.I.-9.

Eighth Embodiment: Comparison of Reaction Time

The following is the experiment for confirmation of required time for chelating reaction.

[Preparation of Reagent and Sample]

The same reagents, the first, second and third reagents, for the chelating reagent, the buffer and the eluant as ones for the first embodiment were used. The Standard Substance of River Water (JAC0031) of the Japan Society for Analytical Chemistry was used by itself as a measurement sample.

[Measurement of Aluminum Concentration]

The chelating reagent, the first reagent, of 50 μL was in a 1.5 mL sample vial, the measurement sample of 300 μL was added therein, and the mixture was swiftly stirred and mixed. The buffer, the second reagent, of 600 μL was added therein and the mixture was swiftly stirred and mixed again. The reaction mixture stood at room temperature (25° C.) for two or 240 minutes and the mixture of 20 μL was injected into the injection portion 18 of the HPLC 10 and measured.

[Condition for HPLC Measurement]

Flow Rate: 1.0 mL/min

Column: Develosil LAL 4.6 mm i.d.×150 mm (Nomura Chemical Co., Ltd.)

Temperature of Column: 40° C.

Wavelength: Ex. (Excitation wavelength)=505 nm, Em. (Detection wavelength)=574 nm

[Result of Measurement]

As shown in Table 4 of the result of measurement, substantially equal values for the retention time, the peak area value and the peak height indicate the completion of chelating reaction in two minutes. Consequently, two or more minutes are suitable for the completion of chelating reaction.

TABLE 4
Comparison of Retention Time, Peak Area Value and Peak Height

| Reaction | Retention | Peak | Peak |
|---|---|---|---|
| Time | Time | Area | Height |
| 2 minutes | 4.46 minutes | 812 × 10⁴ | 300 × 10³ |
| 240 minutes | 4.49 minutes | 804 × 10⁴ | 281 × 10³ |

Ninth Embodiment: Consideration of Kinds of Buffer, or Second Reagent

The following is the experiment for consideration of the kinds of the buffer, or the second reagent.

[Preparation of Reagent and Sample]

The same reagents, the first and third reagents, for the chelating reagent and the eluant as ones for the first embodiment were used. The Standard Substance of River Water (JAC0031) of the Japan Society for Analytical Chemistry was used by itself as a measurement sample.

Buffers of 0.5 mol/L concentration of pH 7.0 were prepared from the respectively following reagents for preparation of a buffer, sodium hydrate (of ultra-high purity) [Kanto Kagaku] and ultrapure water (of ultra-high purity) [Kanto Kagaku].

Reagents for Preparation of Buffer

MES, MOPS, BES, PIPES, HEPES, TES, HEPPSO (2-hydroxy-3-[4-(2-hydroxyethyl)-1-piperazinyl] propanesulfonic acid; monohydrate), EPPS, MOPSO (2-hydroxy-3-morpholinopropanesulfonic acid), Bis-Tris (bis (2-hydroxyethyl) iminotris (hydroxymethyl) methane), ACES (N-(2-acetamido)-2-aminoethanesulfonic acid), ADA (N-(2-acetamido) iminodiacetic acid), CHES, potassium phosphate.

The potassium phosphate is of Wako Pure Chemical Industries, Ltd. and the others are of Dojindo Laboratories.

[Measurement of Aluminum Concentration and Condition for HPLC Measurement]

The aluminum concentration was measured along the lines in the first embodiment except no addition of the measurement sample. Consequently, the measurement provides blank values. And it was also measured in the same HPLC condition as in the first embodiment.

[Result of Measurement]

As shown in Table 5 of the result of measurement, such buffers respectively using MES, MOPS, PIPES, HEPES, TES, EPPS, CHES as a reagent for preparation of the buffer provide reliable measurement of the aluminum concentration with a small blank value measured.

TABLE 5

| Reagent for Preparation of Buffer | Measured Value (ppb) |
|---|---|
| MES | 1.5 |
| MOPS | 3.2 |
| BES | 6.0 |
| PIPES | 1.4 |
| HEPES | 3.7 |
| TES | 2.0 |
| HEPPSO | 4.2 |
| EPPS | 2.4 |
| MOPSO | 9.4 |
| Bis-Tris | 127 |
| ACES | 4.9 |
| ADA | 4.8 |
| CHES | 2.1 |
| Potassium Phosphate | 121 |

Tenth Embodiment: Application to HPLC Using Mobile Phase Solvent in Which Concentration of Organic Solvent is 5% or Less

[Preparation of Reagent and Sample]

The same reagents, the first and second reagents, for the chelating reagent and the buffer as ones for the first embodiment were used. The same 0.1 mol/L acetic acid buffer (pH 4.6) as the third reagent except without 2-propanol was prepared.

The measurement sample of 30 w/v % glucose (Wako Pure Chemical Industries, Ltd) diluted with ultrapure water was used.

[Measurement of Aluminum Concentration]

The aluminum concentration was measured along the lines in the first embodiment.

[Condition for HPLC Measurement]

Flow Rate: 0.5 mL/min

Column: Develosil NPS 4.0 mm i.d.×10 mm (Nomura Chemical Co., Ltd.)

Temperature of Column: 40° C.

Wavelength: Ex. (Excitation wavelength)=505 nm, Em. (Detection wavelength)=574 nm

[Result of Measurement]

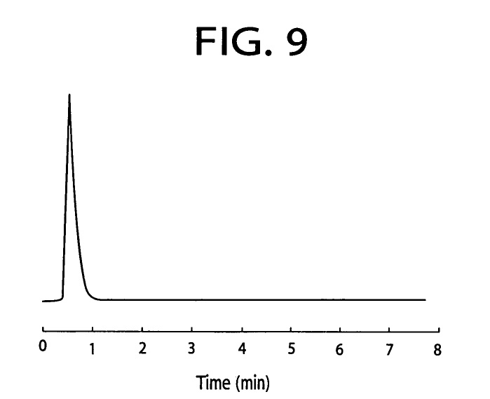

The chromatogram of FIG. 9 illustrates the detected peak of lumogallion-aluminum chelate. The aluminum concentration of 0.8 ppb was calculated from the peak intensity, the reference peak intensity providing blank values and the calibration graph of FIG. 2.

Thus it was found that the lumogallion-aluminum chelate could be detected even if the mobile phase solvent includes no organic solvent. Consequently, the mobile phase solvent with the concentration of 5% or less of the organic solvent is presumed to allow the measurement of aluminum concentration.

While the invention has been described in its exemplary embodiment, the invention may be otherwise embodied.

While the column of 4.6 mm in internal diameter and 150 mm in length suitable for the injected sample amount of 20 μL was employed in the above embodiments according to the invention, the injected sample amount of about 2 μL may allow the measurement of the aluminum concentration in the sample owing to high sensitivity in measurement by the method according to the invention. In this case, it may be measured with employing a column of 0.5-2.0 mm in internal diameter and setting the flow rate as 0.1 mL/min suitable for the amount of the injected sample of about 2 μL.

While the reaction mixture, the mixedly prepared sample, of 950 μL was prepared from the measurement sample of 300 μL, the chelating reagent, the first reagent, of 50 μL and the buffer, the second reagent, of 600 μL in most of the above embodiments, the reaction mixture of increasing volume of the measurement sample, the first reagent and the second reagent in proportion may allow more accurate measurement of the aluminum concentration in the measurement sample owing to substantially preventing the influences or contamination of aluminum from a container or the external environment. The increasing volume may adopt 10 mL or 10 times or more the amount of the reaction mixture in the above embodiments.

It is to be understood that the present invention may be embodied with other changes, improvements, and modifications that may occur to a person skilled in the art without departing from the scope and spirit of the invention defined in the appended claims.

1-8 cancelled.

9. A method for measuring a content of aluminum by detecting aluminum chelate with a fluorescent detector;
wherein the aluminum chelate is generated by reaction in a reaction mixture of a sample containing aluminum and a chelating reagent containing lumogallion, said reaction mixture has a pH value of six or higher.

10. A method for measuring a content of aluminum by detecting aluminum chelate with a fluorescent detector;
wherein the aluminum chelate is generated by reaction in a reaction mixture of a sample containing aluminum and a chelating reagent containing lumogallion and separated from the reaction mixture by liquid chromatography, said reaction mixture has a pH value of six or higher.

11. A method for measuring a content of aluminum according to claim 10, wherein a concentration of an organic solvent in a mobile phase is five percent or less in the liquid chromatography.

12. A method for measuring a content of aluminum according to claim 9,
wherein the reaction mixture contains one of reagents for preparing a buffer, said one of the reagents is selected from a group of 2-morpholinoethanesulfonic acid (MES), piperazine-1,4-bis (2-ethanesulfonic acid) (PIPES), (2-[4-(2-hydroxyethyl)-1-piperazinyl] ethanesulfonic acid (HEPES), N-tris (hydroxymethyl) methyl-2-aminoethanesulfonic acid (TES), (3-[4-(2-hydroxyethyl)-1-piperazinyl] propanesulfonic acid (EPPS), N-cyclohexyl-2-aminoethanesulfonic acid (CHES) and 3-morpholinopropanesulfonic acid (MOPS).

13. A method for measuring a content of aluminum according to claim 9, wherein the reaction mixture is the sample diluted 10-fold or more.

14. A method for measuring a content of aluminum according to claim 9, wherein the sample is a pharmaceutical.

15. A method for measuring a content of aluminum according to claim 14, wherein the pharmaceutical contains vitamin B₂.

16. A method for measuring a content of aluminum according to claim 14, wherein the pharmaceutical contains lipid emulsion.
